(19) United States
(12) Patent Application Publication
(10) Pub. No.: US 2017/0281212 A1
(43) Pub. Date: Oct. 5, 2017

(54) METHODS AND DEVICES FOR LOCALIZING, DELINEATING AND ANCHORING LIVER LESIONS FOR RESECTION

(71) Applicant: Mayo Foundation for Medical Education and Research, Rochester, MN (US)

(72) Inventor: Horacio J. Asbun, Ponte Vedra Beach, FL (US)

(73) Assignee: Mayo Foundation for Medical Education and Research, Rochester, MN (US)

(21) Appl. No.: 15/508,264

(22) PCT Filed: Aug. 26, 2015
(86) PCT No.: PCT/US15/46954

§ 371 Date: Mar. 2, 2017

Related U.S. Application Data
(60) Provisional application No. 62/044,554, filed on Sep. 2, 2014.

(51) Int. Cl. A61B 17/3205; A61B 5/00; A61B 90/00

(52) U.S. Cl. CPC A61B 17/3205; A61B 90/39; A61B 5/4887; A61B 2017/320052

(57) ABSTRACT

This document provides methods and devices for localizing, delineating and/or anchoring lesions for resection from a solid organ (e.g., liver, breast, or pancreas). For example, this document provides devices that can be positioned within liver tissue to help delineate liver tumor tissue from normal liver tissue and to help hold liver tumor tissue in position for resection.

### CROSS REFERENCE TO RELATED APPLICATIONS

This application claims the benefit of U.S. Provisional Ser. No. 62/044,554 filed Sep. 2, 2014. This disclosure of the prior application is considered part of (and is incorporated by reference in) the disclosure of this application.

### BACKGROUND

### 1. Technical Field

This document relates to methods and devices for localizing, delineating, and/or anchoring liver or other organ lesions (e.g., liver tumors) for resection. For example, this document provides devices that can be positioned within liver tissue to help locate a liver tumor with normal liver tissue.

### 2. Background Information

Some liver conditions are effectively treated via surgical resection of the liver lesion. For example, patients presenting with liver metastases, liver cancer, or benign liver tumors can be effectively treated by removing the liver lesion surgically. In these cases, it can be important to remove not only the lesion itself but a region of normal healthy liver tissue that surrounds the lesion. For example, when removing metastatic liver tumor tissue, it can be important to remove a 2 to 10 millimeter margin of healthy normal tissue around the lesion itself.

During image guided surgery (e.g., augmented reality surgery) of organs such as the liver, the imaging taken prior to the operation can be difficult to follow reliably or to superimpose throughout the case to the operative field because of changes during surgery. These changes are secondary to respiratory motion, as well as changes of the relationship of the mass to neighboring tissues because of distortion caused by the actual operation (e.g., retraction of organs, movement secondary to parenchymal resection, etc.).

### SUMMARY

This document provides methods and devices for localizing, delineating, and/or anchoring lesions of the liver (e.g., liver tumors) or other organs (e.g., breast or pancreas tumors) for resection. For example, this document provides devices that can be positioned within tumors within liver tissue to help localize and/or delineate liver tumor tissue from normal liver tissue and to help hold and/or manipulate liver tumor tissue in position for resection.

Solid organ lesions (e.g., liver tumors) that are not located near the surface of the organ can be difficult to resect. For example, internal liver lesions can be located for resection using imaging studies (e.g., ultrasound or other imaging techniques), but the lines of resection for maintaining a proper margin of normal tissue around the lesion can be difficult. In addition, liver lesions within the depths of the liver parenchyma can be difficult to manipulate, and repeated ultrasound examinations may be necessary.

As described herein, the methods and devices provided herein can be used to localize and/or anchor liver lesions (e.g., liver tumors) for resection. For example, the methods and devices provided herein can be used to identify the regions of a liver lesion within the liver and to hold those regions in position to facilitate tissue resection that maintains proper negative margins and preserves healthy liver tissue (e.g., parenchyma) that does not need to be resected. In some cases, the devices provided herein can be introduced before surgery by interventional radiology or can be introduced intraoperatively under imaging guidance before starting the resection.

The devices provided herein can include a hollow shaft portion (e.g., a tube defining a lumen) that can be punctured into or through the lesion such that, for example, a distal end of the shaft portion stays in the center of the lesion or exits the lesion and extends slightly past a lesion/healthy tissue interface (e.g., about 2 mm to about 10 mm, about 3 mm to about 10 mm, about 4 mm to about 10 mm, about 5 mm to about 10 mm, about 6 mm to about 10 mm, about 7 mm to about 10 mm, about 2 mm to about 8 mm, about 2 mm to about 6 mm, about 2 mm to about 6 mm, about 2 mm to about 4 mm, about 3 mm to about 8 mm, or about 4 mm to about 6 mm past a lesion/healthy tissue interface). In some cases, the hollow shaft portion can be punctured through the longest straight path of the lesion such that the distal end of the shaft portion exits the lesion and extends slightly past a lesion/healthy tissue interface (e.g., about 2 mm to about 10 mm, about 3 mm to about 10 mm, about 4 mm to about 10 mm, about 5 mm to about 10 mm, about 6 mm to about 10 mm, about 7 mm to about 10 mm, about 2 mm to about 8 mm, about 2 mm to about 6 mm, about 2 mm to about 6 mm, about 2 mm to about 4 mm, about 3 mm to about 8 mm, or about 4 mm to about 6 mm past a lesion/healthy tissue interface). In some cases, the distal end of the shaft can remain inside the lesion. Once the distal end of the shaft portion is in position, two or more lateral extenders (e.g., two, three, four, five, six, seven, eight, or more lateral extenders) can be deployed from the hollow shaft portion in a radial manner (e.g., from about 45° to about 135° with respect to a longitudinal axis defined by the length-wise direction of the hollow shaft portion) such that, for example, the distal ends of the lateral extenders stay inside the lesion or are slightly extended past the lateral edges of the lesion (e.g., about 2 mm to about 10 mm, about 3 mm to about 10 mm, about 4 mm to about 10 mm, about 5 mm to about 10 mm, about 6 mm to about 10 mm, about 7 mm to about 10 mm, about 2 mm to about 8 mm, about 2 mm to about 6 mm, about 2 mm to about 6 mm, about 2 mm to about 4 mm, about 3 mm to about 8 mm, or about 4 mm to about 6 mm past the lateral edges of the lesion). Once the two or more lateral extenders are in position, the lesion can be surgically resected using the location of the device as a guide to facilitate lesion (e.g., tumor) manipulation and/or tissue resection that maintains proper negative margins and preserves healthy liver tissue (e.g., parenchyma) that does not need to be resected.

In some cases, one or more of the lateral extenders can define a lumen. In such cases, an additional extender can be deployed from the lumen of each of the one or more lateral extenders along a path within healthy liver tissue along an outside edge of the lesion to be resected, for example, in a direction generally toward a surface where the device initially entered the lesion. For example, an additional extender can be deployed from the lumen of each of the one or more lateral extenders to form a cage-like structure around the outside edges of the lesion to be resected. Once the one or more additional extenders are in position, the lesion can be surgically resected using the location of the device as a guide to facilitate tissue resection that maintains proper negative margins and preserves healthy liver tissue (e.g., parenchyma) that does not need to be resected.

In general, one aspect of this document features a device for localizing or delineating a lesion having proximal, distal, and lateral surfaces for resection from a solid organ. The device comprises, or consists essentially of, (a) an elongated shaft portion having a distal end portion and defining a lumen that extends at least a part of the length of the elongated shaft portion, wherein the length of the distal end portion defines a longitudinal axis, wherein the distal end portion of the elongated shaft portion is configured to be advanced into the proximal surface of the lesion, to be advanced through the lesion, and to exit the lesion from the distal surface, (b) two or more extender elements having a distal end portion, wherein the two or more extender elements are configured to be advanced within the lumen of the shaft portion, wherein the distal end portion of the two or more extender elements are configured to exit the lumen at the distal end portion of the elongated shaft portion and are configured to extend away from the distal end portion of the elongated shaft portion along at least a portion of the distal surface of the lesion in a direction between about 45 degrees and about 135 degrees with respect to the longitudinal axis, and wherein at least one of the two or more extender elements defines a lumen, and (c) an additional extender element having a distal end portion, wherein the additional extender element is configured to be advanced within the lumen of the at least one extender element defining the lumen, wherein the distal end portion of the additional extender element is configured to exit the lumen of the extender element defining the lumen at the distal end portion of the extender element defining the lumen and is configured to extend away from the distal end portion of the extender element defining the lumen along at least a portion of the lateral surface of the lesion in a direction between about 45 degrees and about 135 degrees with respect to a longitudinal axis of the extender element defining the lumen. The lesion can be a liver tumor. The elongated shaft portion can comprise a tubular element. The device can comprise two extender elements. The device can comprise three extender elements. The device can comprise four extender elements. The device can comprise five extender elements. The device can comprise six extender elements. The two or more extender elements can be configured to extend away from the distal end portion of the elongated shaft portion along at least a portion of the distal surface of the lesion in a direction between about 55 degrees and about 125 degrees with respect to the longitudinal axis. The two or more extender elements can be configured to extend away from the distal end portion of the elongated shaft portion along at least a portion of the distal surface of the lesion in a direction between about 65 degrees and about 115 degrees with respect to the longitudinal axis. The two or more extender elements can be configured to extend away from the distal end portion of the elongated shaft portion along at least a portion of the distal surface of the lesion in a direction between about 75 degrees and about 105 degrees with respect to the longitudinal axis. The two or more extender elements can be configured to extend away from the distal end portion of the elongated shaft portion along at least a portion of the distal surface of the lesion in a direction between about 85 degrees and about 95 degrees with respect to the longitudinal axis. Each of the two or more extender elements can be configured to extend away from the distal end portion of the elongated shaft portion in a different radial direction along at least a portion of the distal surface of the lesion. The two or more extender elements can comprise shape memory material. The shape memory material can be nitinol. Each of the two or more extender elements can define a lumen. The device can comprise one of the additional extender elements for each of the two or more extender elements. The distal end portion of the additional extender element can be configured to extend away from the distal end portion of the extender element defining the lumen along at least a portion of the lateral surface of the lesion in a direction between about 65 degrees and about 115 degrees with respect to a longitudinal axis of the extender element defining the lumen. The distal end portion of the additional extender element can be configured to extend away from the distal end portion of the extender element defining the lumen along at least a portion of the lateral surface of the lesion in a direction between about 75 degrees and about 105 degrees with respect to a longitudinal axis of the extender element defining the lumen. The distal end portion of the additional extender element can be configured to extend away from the distal end portion of the extender element defining the lumen along at least a portion of the lateral surface of the lesion in a direction between about 85 degrees and about 95 degrees with respect to a longitudinal axis of the extender element defining the lumen. The additional extender element can comprise shape memory material. The shape memory material can be nitinol.

In another aspect, this document features a method for localizing or delineating at least a portion of a lesion for resection from a solid organ. The method comprises, or consists essentially of, (a) advancing an elongated shaft portion having a distal end portion and defining a lumen that extends at least a part of the length of the elongated shaft portion into a proximal surface of a lesion and through the lesion until about 2 mm to about 10 mm of the distal end portion of the elongated shaft portion exits a distal surface of the lesion, and (b) advancing two or more extender elements having a distal end portion within the lumen of the shaft portion in a manner wherein each distal end portion of the two or more extender elements extends away from the distal end portion of the shaft portion along at least a portion of the distal surface of the lesion until a distance about 2 mm to about 10 mm past a lateral edge of the lesion is reached, thereby delineating at least a portion of the lesion for resection. The lesion can be a liver tumor. The elongated shaft portion can comprise a tubular element. The method can comprise advancing two extender elements. The method can comprise advancing three extender elements. The method can comprise advancing four extender elements. The method can comprise advancing five extender elements. The method can comprise advancing six extender elements. The method can comprise extending the distal end portions of the two or more extender elements away from the distal end portion of the elongated shaft portion along at least a portion of the distal surface of the lesion in a direction between about 45 degrees and about 135 degrees with respect to a longitudinal axis of the distal end portion of the elongated shaft portion. The method can comprise extending the distal end portions of the two or more extender elements away from the distal end portion of the elongated shaft portion along at least a portion of the distal surface of the lesion in a direction between about 55 degrees and about 125 degrees with respect to a longitudinal axis of the distal end portion of the elongated shaft portion. The method can comprise extending the distal end portions of the two or more extender elements away from the distal end portion of the elongated shaft portion along at least a portion of the distal surface of the lesion in a direction between about 65 degrees and about 115 degrees with respect to a longitudinal axis of the distal end portion of the elongated shaft portion. The method can comprise extending the distal end portions of the two or more extender elements away from the distal end portion of the elongated shaft portion along at least a portion of the distal surface of the lesion in a direction between about 75 degrees and about 105 degrees with respect to a longitudinal axis of the distal end portion of the elongated shaft portion. The method can comprise extending the distal end portions of the two or more extender elements away from the distal end portion of the elongated shaft portion along at least a portion of the distal surface of the lesion in a direction between about 85 degrees and about 95 degrees with respect to a longitudinal axis of the distal end portion of the elongated shaft portion. Each of the two or more extender elements can be configured to extend away from the distal end portion of the elongated shaft portion in a different radial direction along at least a portion of the distal surface of the lesion. The two or more extender elements can comprise shape memory material. The shape memory material can be nitinol. At least one of the two or more extender elements can define a lumen. Each of the two or more extender elements can define a lumen. The method can comprise advancing an addition extender element having a distal end portion within the lumen of the extender element in a manner wherein the distal end portion of the additional extender element extends away from the distal end portion of the extender element along at least a portion of a lateral surface of the lesion. The method can comprise extending the distal end portion of the additional extender element until a distance about half the overall length of the lesion is reached. The method can comprise advancing one of the additional extender elements for each of the two or more extender elements. The distal end portion of the additional extender element can extend away from the distal end portion of the extender element along at least a portion of the lateral surface of the lesion in a direction between about 45 degrees and about 135 degrees with respect to a longitudinal axis of the extender element. The distal end portion of the additional extender element can extend away from the distal end portion of the extender element along at least a portion of the lateral surface of the lesion in a direction between about 65 degrees and about 115 degrees with respect to a longitudinal axis of the extender element. The distal end portion of the additional extender element can extend away from the distal end portion of the extender element along at least a portion of the lateral surface of the lesion in a direction between about 75 degrees and about 105 degrees with respect to a longitudinal axis of the extender element. The distal end portion of the additional extender element can extend away from the distal end portion of the extender element along at least a portion of the lateral surface of the lesion in a direction between about 85 degrees and about 95 degrees with respect to a longitudinal axis of the extender element. The additional extender element can comprise shape memory material. The shape memory material can be nitinol. The method can comprise resecting the lesion.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a side view of a device for localizing, delineating, and/or anchoring a liver lesion for resection, in accordance with one embodiment provided herein.

FIG. 2 is a side view of the device of FIG. 1 with six extender elements deployed, in accordance with one embodiment provided herein.

FIG. 3 is a cross-sectional view of the device of FIG. 1, in accordance with one embodiment provided herein.

FIG. 4 is a cross-sectional view of a device similar to that of FIG. 1 except that the diameter of the deployment shaft for the extender elements is larger, in accordance with one embodiment provided herein.

FIG. 5 is a cross-sectional view of a device for localizing, delineating, and/or anchoring a liver lesion for resection, in accordance with one embodiment provided herein.

FIG. 6 is a cross-sectional view of a device for localizing, delineating, and/or anchoring a liver lesion for resection, in accordance with one embodiment provided herein.

FIG. 7 is a side view of the device of FIG. 6 with three extender elements deployed, in accordance with one embodiment provided herein.

FIG. 8 is a side view of a device for localizing, delineating, and/or anchoring a liver lesion for resection, in accordance with one embodiment provided herein.

FIG. 9 is a side view of the device of FIG. 1 with six extender elements deployed in relationship to a liver lesion, in accordance with one embodiment provided herein.

FIG. 10 is a side view of the device of FIG. 8 with extender elements deployed in relationship to a liver lesion, in accordance with one embodiment provided herein.

FIG. 11 is a side view of a device with six extender elements and six additional extender elements deployed in relationship to a liver lesion, in accordance with one embodiment provided herein.

### DETAILED DESCRIPTION

This document provides methods and devices for localizing, delineating, and/or anchoring liver lesions or lesions of other organs (e.g., breast tumors or pancreas tumors) for resection. For example, this document provides devices that can be positioned within liver tissue to help localize or delineate liver tumor tissue from normal liver tissue and to help hold liver tumor tissue in position or manipulate the liver tumor tissue for resection.

In general, the devices provided herein can include a hollow shaft portion (e.g., a tube defining a lumen) that can be punctured into or through the lesion such that, for example, a distal end of the shaft portion stays within the lesion (e.g., within the center of the lesion) or exits the lesion and extends slightly past a lesion/healthy tissue interface (e.g., about 2 mm to about 10 mm, about 3 mm to about 10 mm, about 4 mm to about 10 mm, about 5 mm to about 10 mm, about 6 mm to about 10 mm, about 7 mm to about 10 mm, about 2 mm to about 8 mm, about 2 mm to about 6 mm, about 2 mm to about 6 mm, about 2 mm to about 4 mm, about 3 mm to about 8 mm, or about 4 mm to about 6 mm past a lesion/healthy tissue interface). In some cases, the hollow shaft portion can be punctured through the longest straight path of lesion such that the distal end of the shaft portion exits the lesion and extends slightly past a lesion/healthy tissue interface (e.g., about 2 mm to about 10 mm, about 3 mm to about 10 mm, about 4 mm to about 10 mm, about 5 mm to about 10 mm, about 6 mm to about 10 mm, about 7 mm to about 10 mm, about 2 mm to about 8 mm, about 2 mm to about 6 mm, about 2 mm to about 6 mm, about 2 mm to about 4 mm, about 3 mm to about 8 mm, or about 4 mm to about 6 mm past a lesion/healthy tissue interface). In some cases, the distal end of the shaft portion can remain inside the bulk of the lesion.

Once the distal end of the shaft portion is in position, two or more extender elements (e.g., two, three, four, five, six, seven, eight, or more lateral extender elements) can be deployed from the hollow shaft portion in a generally radial manner (e.g., from about 45° to about 135° with respect to a longitudinal axis defined by the length-wise direction of the hollow shaft portion) such that, for example, the distal ends of the extender elements are inside the lesion or are slightly past the lateral edges of the lesion (e.g., about 2 mm to about 10 mm, about 3 mm to about 10 mm, about 4 mm to about 10 mm, about 5 mm to about 10 mm, about 6 mm to about 10 mm, about 7 mm to about 10 mm, about 2 mm to about 8 mm, about 2 mm to about 6 mm, about 2 mm to about 6 mm, about 2 mm to about 4 mm, about 3 mm to about 8 mm, or about 4 mm to about 6 mm past the lateral edges of the lesion). Once the two or more extender elements are in position, the lesion can be surgically resected using the location of the device as a guide to facilitate tissue resection that maintains proper negative margins and preserves healthy liver tissue (e.g., parenchyma) that does not need to be resected.

In some cases, the hollow shaft portion can be punctured through the lesion such that, for example, a distal end of the shaft portion remains within the lesion. In these cases, at least a portion of the two or more extender elements can be advanced to exit the lesion and extend slightly past a lesion/healthy tissue interface before being advanced radially.

In some cases, one or more of the extender elements can define a lumen. For example, the extender elements can be in the form of a tube or a coil (e.g., a nitinol coil) that defines a lumen. In such cases, an additional extender element can be deployed from the lumen of each of the one or more extender elements along a path within healthy liver tissue along an outside edge of the lesion to be resected, for example, in a direction generally toward a surface where the device initially entered the lesion. For example, an additional extender element can be deployed from the lumen of each of the one or more extender elements to form a cage-like structure around the outside edges of the lesion to be resected. Once the one or more additional extender elements are in position, the lesion can be surgically resected using the location of the device as a guide to facilitate tissue resection that maintains proper negative margins and preserves healthy liver tissue (e.g., parenchyma) that does not need to be resected.

In some cases, a device provided herein can serve as a marker guide (e.g., a fiduciary to guide resection). For example, in augmented reality surgery, the imaging taken prior to the operation can be difficult to superimpose reliably throughout the case to the operative field because of changes during surgery. These changes can be secondary to respiratory motion, as well as changes of the relationship of the mass to neighboring tissues because of distortion caused by the actual operation (e.g., retraction of organs, movement secondary to parenchymal resection, etc.). A device provided herein can have different type of materials or include a small marker that would is easy to detect intraoperatively either by imaging or other similar localization techniques (e.g., radioactive or magnetic), allowing for the detection of the device and re-alignment of coordinates to guide the resection. This can allow a surgeon to follow the lesion's location and its relationship to neighboring structures throughout the surgical procedure regardless of the changes afflicted by the actual surgery, the patient's position, or breathing motion.

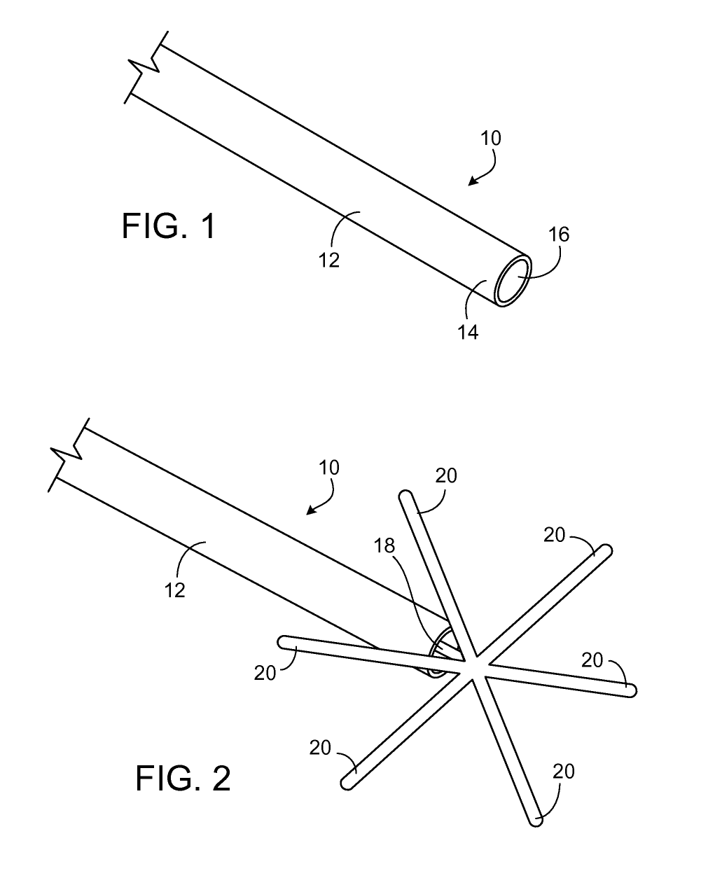

With reference to FIGS. 1 and 2, a device 10 for localizing, delineating, and/or anchoring a liver lesion can include an elongated shaft portion 12 having a distal end portion 14 and defining a lumen 16. Device 10 can include two or more extender elements 20 (e.g., lateral extender elements). For example, device 10 can include two, three, four, five, six, seven, eight, nine, ten, or more extender elements 20. Extender elements 20 can be deployed from lumen 16 to extend away from distal end portion 14 at an angle that is from about 45° to about 135° (e.g., from about 55° to about 125°, from about 65° to about 115°, from about 75° to about 105°, or from about 85° to about 95° with respect to a longitudinal axis defined by the length-wise direction of distal end portion 14. In some cases, this angle can be about 90°. In some cases, extender elements 20 can be attached to a shaft 18.

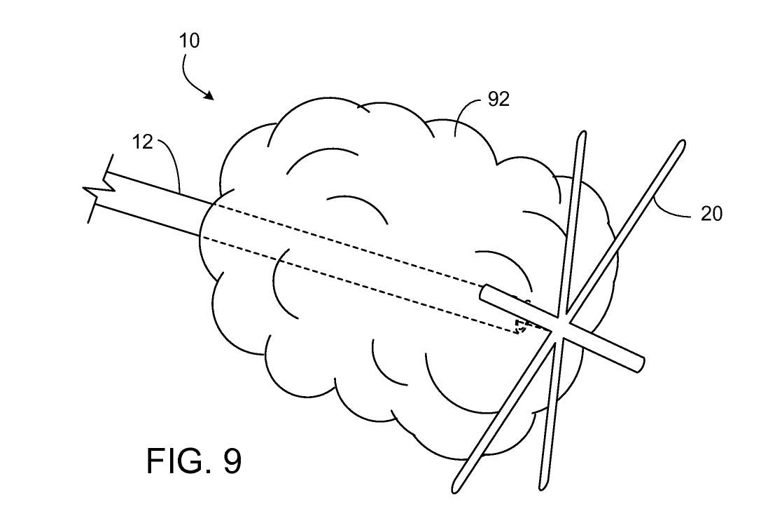

When device 10 of FIG. 2 is deployed within liver tissue having liver lesion 92 as shown in FIG. 9, device 10 helps localize and/or delineate liver lesion 92 from surrounding normal liver tissue and helps hold liver lesion 92 in position for resection.

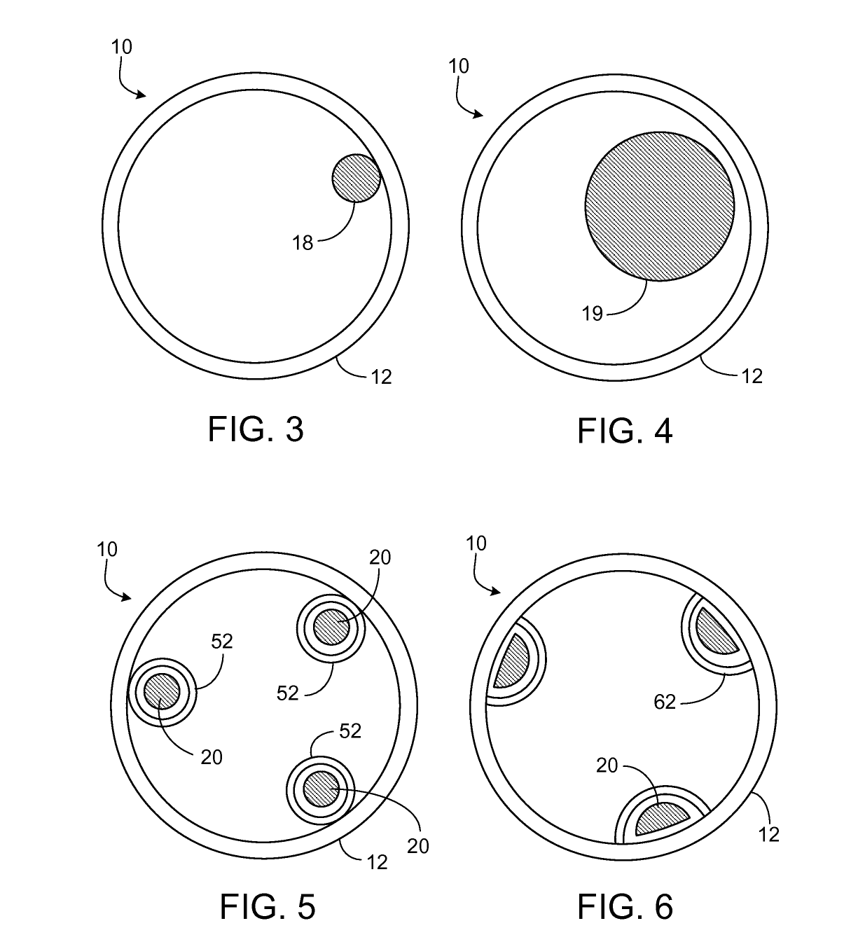

With reference to FIGS. 3 and 4, the cross-sectional size (e.g., diameter) of shaft 18 (or the cross-sectional size or diameter of the two or more extender elements 20) can be relatively small (e.g., less than 20, 15, 10, or 5 percent of the cross-sectional size of lumen 16) or can be somewhat larger (e.g., between about 20 percent and about 95 percent, between about 35 percent and about 90 percent, between about 50 percent and about 90 percent, between about 75 percent and about 90 percent, or between about 50 percent and about 75 percent of the cross-sectional size of lumen 16). In some cases, the cross-sectional size (e.g., diameter) of shaft 18 (or the cross-sectional size or diameter of the two or more extender elements 20) can be slightly smaller than the cross-sectional size of lumen 16 (e.g., about 1 to 5 percent smaller than the cross-sectional size of lumen 16).

With reference to FIG. 5, device 10 can include inner channels 52 configured to define a lumen for extender elements 20. In these cases, each extender element 20 can be deployed from its own inner channel 52. The lumen of inner channels 52 can be any appropriate shape. For example, the lumen of inner channels 52 can be circular. In some cases, device 10 can include inner channels 62 configured to define an asymmetrical shaped lumen (e.g., crescent-shaped lumen) for extender elements 20 (FIG. 6). In these cases, each extender element 20 can be deployed from its own inner channel 62. In addition, extender elements 20 can be shaped to deploy from an asymmetrical shaped lumen without rotating or spinning to aid in maintaining the direction each extender element 20 is to be advanced.

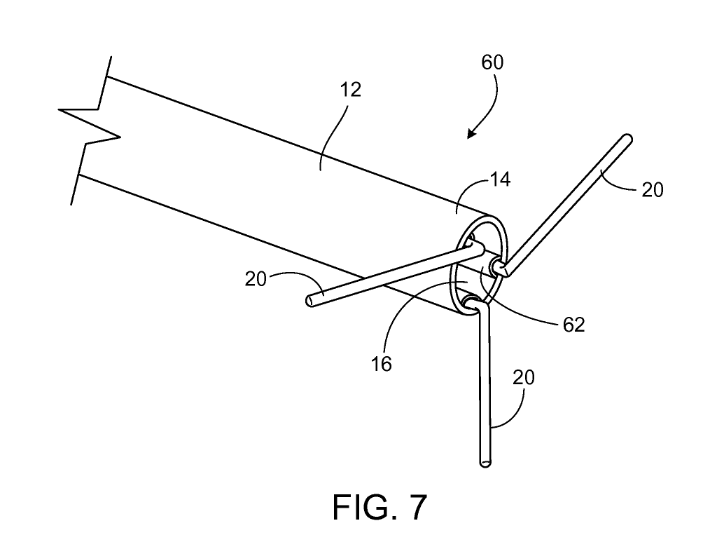

With reference to FIG. 7, device 60 can include shaft portion 12 that defines lumen 16 and includes distal end portion 14 and two or more inner channels 62. For example, device 60 can include two, three, four, five, six, seven, eight, nine, ten, or more inner channels 62. Device 60 can include extender elements 20. For example, device 60 can include an extender element 20 for each inner channel 62. Extender elements 20 can be deployed from distal end portion 14 to extend away from distal end portion 14 at an angle that is from about 45° to about 135° (e.g., from about 55° to about 125°, from about 65° to about 115°, from about 75° to about 105°, or from about 85° to about 95°) with respect to a longitudinal axis defined by the length-wise direction of distal end portion 14. In some cases, this angle can be about 90°.

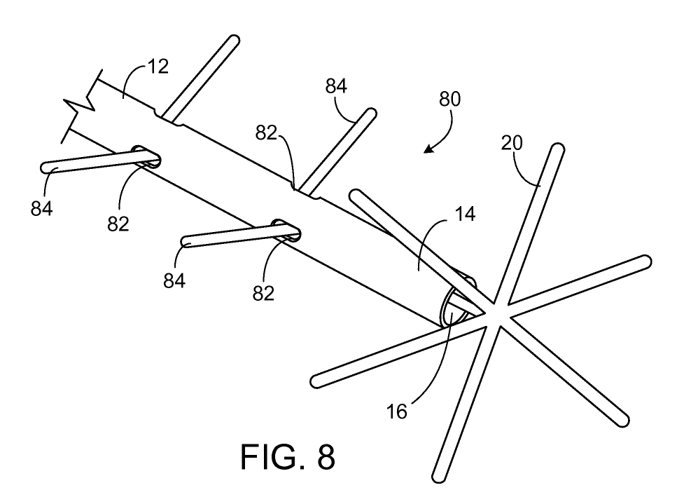

With reference to FIG. 8, device 80 can include shaft portion 12 that defines lumen 16 and includes distal end portion 14 and two or more extender elements 20. For example, device 60 can include an extender element 20 for each inner channel 62. Extender elements 20 can be deployed from distal end portion 14 to extend away from distal end portion 14 at an angle that is from about 45° to about 135° (e.g., from about 55° to about 125°, from about 65° to about 115°, from about 75° to about 105°, or from about 85° to about 95°) with respect to a longitudinal axis defined by the length-wise direction of distal end portion 14. In some cases, this angle can be about 90°. In some cases, device 80 can include one or more openings 82 (e.g., one, two, three, four, five, six, seven, eight, nine, ten, or more openings 82) and one or more shaft extenders 84 (e.g., one, two, three, four, five, six, seven, eight, nine, ten, or more shaft extenders 84) configured to deploy from lumen 16 of shaft portion 12 via openings 82. For example, device 80 can include one shaft extenders 84 for each opening 82. In some cases, device 80 can include one or more rows (e.g., two, three, four, five, or more rows) of two or more openings 82 (e.g., two, three, four, five, six, seven, eight, nine, ten, or more openings 82) at a given length from distal end portion 14. For example, device 80 can include two rows of three openings 80, where the distal row is between about 4 mm and about 10 mm from the distal end of distal end portion 14 and the proximal row is between about 4 mm and about 10 mm from the distal row.

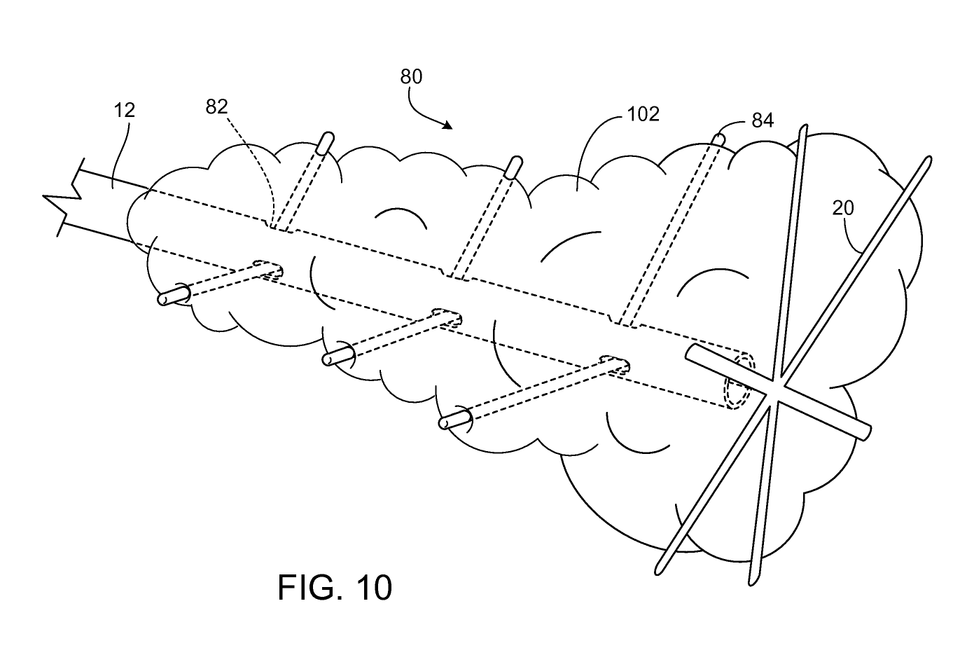

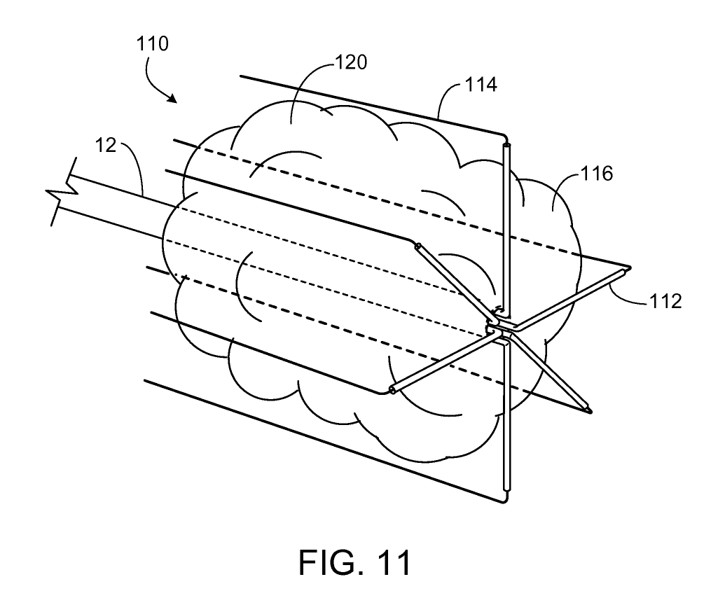

When device 80 is deployed within liver tissue having liver lesion 102 as shown in FIG. 10, device 80 helps localize and/or delineate liver lesion 102 from surrounding normal liver tissue and helps hold and/or manipulate liver lesion 102 in position for resection.

With reference to FIG. 11, device 110 can include shaft portion 12 that defines a lumen and includes a distal end portion and two or more extender elements 112. In some cases, one or more of extender elements 112 can define a lumen. For example, each extender element 112 of device 110 can define a lumen. Device 110 can include one or more additional extender elements 114. Each additional extender element 114 can be deployed from a distal end portion of an extender element 112 to extend away from the distal end portion of that extender element 112 at an angle that is from about 45° to about 135° (e.g., from about 55° to about 125°, from about 65° to about 115°, from about 75° to about 105°, or from about 85° to about 95°) with respect to a longitudinal axis defined by the length-wise direction of that extender element 112. In some cases, this angle can be about 90°.

When device 110 is deployed within liver tissue having liver lesion 120 as shown in FIG. 11, device 110 helps localize and/or delineate liver lesion 120 from surrounding normal liver tissue and helps hold and/or manipulate liver lesion 120 in position for resection.

The shaft portion and inner channels of a device provided herein can be made of any appropriate material. For example, the shaft portion and inner channels of a device provided herein can be made of a biocompatible material such as a plastic, a polymer of a natural material, a polymer of a synthetic material, or metal. Different materials can be used at the tip or in other parts for better detection by imaging and/or localizing techniques. In some cases, the end of the device that it is outside the liver can be attached to a suture type material such as silk, polypropylene, or plastic.

The extender elements, shaft extender elements, and additional extender elements of a device provided herein can be made of any appropriate material. For example, the extender elements, shaft extender elements, and additional extender elements of a device provided herein can include any appropriate shape memory material such as nitinol.

In some cases, the extender elements, shaft extender elements, and additional extender elements of a device provided herein can include markings that allow a user to deploy a predetermined length of the extender elements, shaft extender elements, and additional extender elements into liver tissue. In some cases, exchangeable extender elements, shaft extender elements, and additional extender elements of predetermined lengths can be used such that the proper extension length of each within the liver tissue is achieved. The lengths to be deployed can be determined using imaging techniques for visualizing the liver lesion to be resected such as ultrasound techniques.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A device for localizing or delineating a lesion having proximal, distal, and lateral surfaces for resection from a solid organ, wherein said device comprises:
(a) an elongated shaft portion having a distal end portion and defining a lumen that extends at least a part of the length of said elongated shaft portion, wherein the length of said distal end portion defines a longitudinal axis, wherein said distal end portion of said elongated shaft portion is configured to be advanced into said proximal surface of said lesion, to be advanced through said lesion, and to exit said lesion from said distal surface,
(b) two or more extender elements having a distal end portion, wherein said two or more extender elements are configured to be advanced within said lumen of said shaft portion, wherein said distal end portion of said two or more extender elements are configured to exit said lumen at said distal end portion of said elongated shaft portion and are configured to extend away from said distal end portion of said elongated shaft portion along at least a portion of said distal surface of said lesion in a direction between about 45 degrees and about 135 degrees with respect to said longitudinal axis, and wherein at least one of said two or more extender elements defines a lumen, and
(c) an additional extender element having a distal end portion, wherein said additional extender element is configured to be advanced within said lumen of said at least one extender element defining said lumen, wherein said distal end portion of said additional extender element is configured to exit said lumen of said extender element defining said lumen at said distal end portion of said extender element defining said lumen and is configured to extend away from said distal end portion of said extender element defining said lumen along at least a portion of said lateral surface of said lesion in a direction between about 45 degrees and about 135 degrees with respect to a longitudinal axis of said extender element defining said lumen.

2. The device of claim 1, wherein said lesion is a liver tumor.

3. The device of claim 1, wherein said elongated shaft portion comprises a tubular element.

4-8. (canceled)

9. The device of claim 1, wherein said two or more extender elements are configured to extend away from said distal end portion of said elongated shaft portion along at least a portion of said distal surface of said lesion in a direction between about 55 degrees and about 125 degrees with respect to said longitudinal axis.

10-12. (canceled)

13. The device of claim 1, wherein each of said two or more extender elements is configured to extend away from said distal end portion of said elongated shaft portion in a different radial direction along at least a portion of said distal surface of said lesion.

14. The device of claim 1, wherein said two or more extender elements comprise shape memory material.

15. (canceled)

16. The device of claim 1, wherein each of said two or more extender elements defines a lumen.

17. The device of claim 1, wherein said device comprises one of said additional extender elements for each of said two or more extender elements.

18-20. (canceled)

21. The device of claim 1, wherein said additional extender element comprises shape memory material.

22. (canceled)

23. A method for localizing or delineating at least a portion of a lesion for resection from a solid organ, wherein said method comprises:
(a) advancing an elongated shaft portion having a distal end portion and defining a lumen that extends at least a part of the length of said elongated shaft portion into a proximal surface of a lesion and through said lesion until about 2 mm to about 10 mm of said distal end portion of said elongated shaft portion exits a distal surface of said lesion, and
(b) advancing two or more extender elements having a distal end portion within said lumen of said shaft portion in a manner wherein each distal end portion of said two or more extender elements extends away from said distal end portion of said shaft portion along at least a portion of said distal surface of said lesion until a distance about 2 mm to about 10 mm past a lateral edge of said lesion is reached, thereby delineating at least a portion of said lesion for resection.

24. The method of claim 23, wherein said lesion is a liver tumor.

25. The method of claim 23, wherein said elongated shaft portion comprises a tubular element.

26-30. (canceled)

31. The method of claim 23, wherein said method comprises extending said distal end portions of said two or more extender elements away from said distal end portion of said elongated shaft portion along at least a portion of said distal surface of said lesion in a direction between about 45 degrees and about 135 degrees with respect to a longitudinal axis of said distal end portion of said elongated shaft portion.

32-35. (canceled)

36. The method of claim 23, wherein each of said two or more extender elements is configured to extend away from said distal end portion of said elongated shaft portion in a different radial direction along at least a portion of said distal surface of said lesion.

37. The method of claim 23, wherein said two or more extender elements comprise shape memory material.

38. (canceled)

39. The method of claim 23, wherein at least one of said two or more extender elements defines a lumen.

40. (canceled)

41. The method of claim 39, wherein said method comprises advancing an addition extender element having a distal end portion within said lumen of said extender element in a manner wherein said distal end portion of said additional extender element extends away from said distal end portion of said extender element along at least a portion of a lateral surface of said lesion.

42. The method of claim 41, wherein said method comprises extending said distal end portion of said additional extender element until a distance about half the overall length of said lesion is reached.

43. The method of claim 41, wherein said method comprises advancing one of said additional extender elements for each of said two or more extender elements.

44-49. (canceled)

50. The method of claim 23, wherein said method comprises resecting said lesion.
